(19) United States
(12) Patent Application Publication
(10) Pub. No.: US 2020/0107816 A1
(43) Pub. Date: Apr. 9, 2020

(54) ELECTRICAL INTERCONNECT FOR USE IN AN ULTRASOUND TRANSDUCER

(71) Applicant: General Electric Company, Schenectady, NY (US)

(72) Inventors: Warren Lee, Nisayuna, NY (US); Matthew Karesh, West Chester, OH (US); Craig Mirr, Brookfield, WI (US); Stephen Crynock, Fredonia, WI (US)

(21) Appl. No.: 16/155,602

(22) Filed: Oct. 9, 2018

(51) Int. Cl. A61B 8/00; B06B 1/06; G10K 11/00

(52) U.S. Cl. CPC A61B 8/4494; B06B 1/0629; B06B 2201/76; G10K 11/004; A61B 8/4455

(57) ABSTRACT

The present approach relates to an interconnect structure (e.g., an electrical standoff) for use between two electrical components, such as a matrix transducer array and ASIC of an ultrasound probe and to the manufacture of such a structure. In accordance with certain embodiments, the interconnect structure provides electrical interconnection between electrical components and provides improved acoustic attenuation.

### BACKGROUND

The subject matter disclosed herein relates to an electrical interconnect structure that may be suitable in various electrical devices, such as, but not limited to, an ultrasound transducer.

An ultrasound imaging system typically includes an ultrasound probe that is applied to a patient's body and a workstation or monitor that is operably coupled to the ultrasound probe. The ultrasound probe may be controlled by an operator of the ultrasound imaging system and is configured to transmit and receive ultrasound signals that are processed into an ultrasound image by the workstation or monitor. The operator positions the ultrasound probe to acquire image data of a target anatomy or region of interest (e.g., a desired tissue or body region to be imaged) in a target scan plane.

As ultrasound imaging has become more sophisticated, the quantity of data acquired and the acquisition methodology have placed increasing demands on the data acquisition components and hardware. For example, for electronic 4D (real-time, three-dimensional (3D)) imaging ultrasound probes, a two-dimensional array of thousands of ultrasound transducer elements is used to steer and focus the ultrasound beam over a volumetric field of view. Integrated circuits (ASICs) inside the probe process signals to and from the thousands of transducer elements. Typically, there is an individual electrical connection between each transducer element and the associated electronics in the ASICs. Forming such a number of interconnects in a suitably sized package (e.g., a handheld ultrasound probe) and having suitable electronic properties with respect to noise and signal transmission can be problematic. Current approaches for forming such interconnects are laborious and require high degrees of precision in the manufacture process, which may make manufacture of the probe a time-consuming process that is subject to error.

### BRIEF DESCRIPTION

In one embodiment, an electrical standoff is provided. In accordance with this embodiment, the electrical standoff comprises: a plurality of acoustic backing layers, wherein the acoustic backing layers are stacked on one another to form a laminated structure; and a plurality of conductive traces formed on one or more of the acoustic backing layers, wherein the conductive traces form conductive contacts on different surfaces of the laminated structure for coupling two electrical components.

In a further embodiment, a method is provided for forming an electrical standoff structure. In accordance with this method, conductive ink is printed to form conductive traces on one or more acoustic backing layers of a plurality of acoustic backing layers. The plurality of acoustic backing layers are stacked. The plurality of acoustic backing layers are laminated to form a laminated structure used to form the electrical standoff structure.

In an additional embodiment, an ultrasound probe is provided. In accordance with this embodiment, the ultrasound probe comprises: a transducer array comprising a plurality of separate transducer elements; driving and receiving circuitry configured to communicate with each individual transducer element; and an electrical standoff structure electrically connecting the driving and receiving circuitry for each transducer element to each respective transducer element. The electrical standoff structure comprises a laminated stack of acoustic backing layers on which conductive traces are formed on at least a portion of the acoustic backing layers.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is an embodiment of a block diagram of an ultrasound system, in accordance with aspects of the present disclosure;

FIG. 2 depicts an example of an arrangement of interleaved flex circuit layers and acoustic backing layers used to form an electrical interconnect structure;

FIG. 3 depicts an example of an arrangement of stacked acoustic backing layers, on which traces are printed, used to form an electrical interconnect structure, in accordance with aspects of the present disclosure;

FIG. 4 depicts an end view of an interconnect structure formed using stacked acoustic backing layers as shown in FIG. 3, in accordance with aspects of the present disclosure;

FIG. 5 depicts an example of an arrangement of stacked acoustic backing layers, on which traces are printed on certain acoustic backing layer but not all backing layers, in accordance with aspects of the present disclosure;

FIG. 6 depicts an end view of an interconnect structure formed using stacked acoustic backing layers as shown in FIG. 5, in accordance with aspects of the present disclosure;

FIG. 7 depicts a perspective view of an acoustic backing layer, in accordance with aspects of the present disclosure;

FIG. 8 depicts an end-view of the acoustic backing layer of FIG. 7, in accordance with aspects of the present disclosure;

FIG. 9 depicts an example of an arrangement of stacked acoustic backing layers of FIGS. 7 and 8 interleaved with acoustic backing layers on which no traces are printed, in accordance with aspects of the present disclosure;

FIG. 10 depicts an end view of an interconnect structure formed using stacked acoustic backing layers as shown in FIG. 9, in accordance with aspects of the present disclosure;

FIG. 11 depicts an example of printing multiple acoustic backing layers on a larger sheet, in accordance with aspects of the present disclosure;

FIG. 12 depicts an example of forming a stack of acoustic backing layers using an alignment tool, and with an additional inset view, in accordance with aspects of the present disclosure;

FIG. 13 depicts an example of forming a larger laminated structure that may be cut to form interconnect structures, in accordance with aspects of the present disclosure;

FIG. 14 depicts an acoustic backing sheet in which conductive traces are printed so as to have different spacing at the different ends of the acoustic backing sheet, in accordance with aspects of the present disclosure;

FIG. 15 depicts an acoustic backing sheet in which conductive traces are printed so as to have angled turns along their length, in accordance with aspects of the present disclosure;

FIG. 16 depicts a view of an embodiment of an electrical interconnect structure in which connected devices are not connected on opposing surfaces of the interconnect structure, in accordance with aspects of the present disclosure;

FIG. 17 depicts a view of another embodiment of an electrical interconnect structure in which connected devices are not connected on opposing surfaces of the interconnect structure, in accordance with aspects of the present disclosure; and

FIG. 18 depicts a process flow for manufacturing an interconnect structure in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As ultrasound imaging has become more sophisticated, the quantity of data acquired and the acquisition methodology have placed increasing demands on the data acquisition components and hardware. For example, an imaging probe suitable for 4D imaging may incorporate thousands of individual ultrasound transducer elements. In practice, integrated circuits (ASICs) inside the probe process signals to and from these thousands of transducer elements. Typically, there is an individual electrical connection between each transducer element and the associated electronics in the ASICs.

In conventional approaches, these interconnections are achieved by one of two approaches. In the first approach, alternating sheets of flex circuits and acoustic backing are laminated together to form a “flex-plus-backing” stack that is post-processed to form a two-dimensional grid of interconnects. One end of the laminated stack electrically connects to the matrix array of transducer elements, while the opposite end of the stack interfaces with the electrical elements within the probe handle. In the other approach, the matrix array is directly fabricated on top of the ASIC via bumps or other interconnect structures connected to the respective transducer element.

Both methods have shortcomings. With respect to the “flex-plus-backing” approach, the need for alternating layers of flex circuit and acoustic backing is costly and complicated and the protrusion of the relatively thick (e.g., ˜18 microns thick) low attenuation metal traces on the flex circuits into the acoustic backing degrades the acoustic performance. With respect to the direct fabrication approach, the ASICs and their associated interconnects are significantly larger in size compared to the active acoustic aperture, and therefore the probe footprint is undesirably enlarged. Also, the element spacing in the matrix array must match the spacing of the bumps on the ASIC, which prevents the ASIC from being used in multiple applications where different element spacings are required. Finally, the proximity of the ASIC to transducer array causes the patient contact surface to be heated from power dissipation in the ASIC, which may thermally limit operation of the probe.

In accordance with the present approach, shortcomings such as those noted above are avoided. In particular, the presently disclosed approaches addresses these shortcomings by describing a structure and method of manufacture for an electrical standoff between the matrix transducer array and ASIC (which may be directly coupled to the present electrical standoff structure or coupled via an intermediary, such as a separate flex interconnect circuit) which provides electrical interconnection and improved acoustic attenuation. The standoff also allows for a reduced probe footprint, the ability to have independent ASIC bump and element spacing, and potentially improved thermal performance. In addition, the standoff can be manufactured in a way that is simpler and more cost-effective than previous methods.

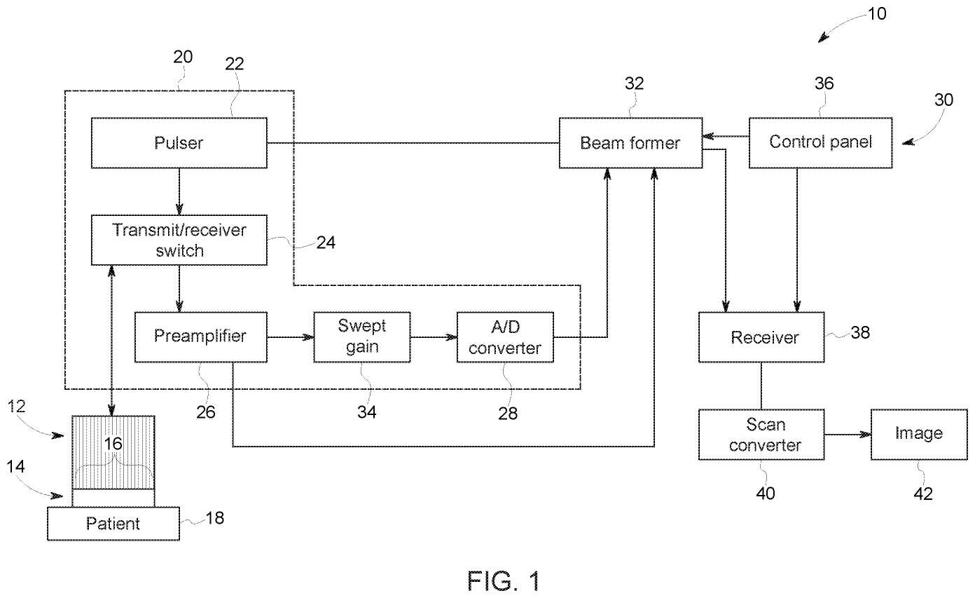

With the preceding in mind, and by way of providing useful context, FIG. 1 depicts a high-level view of components of an ultrasound system 10 that may be employed in accordance with the present approach. In particular, the electrical interconnect structure 12 disclosed herein function as an “electrical standoff” that electrically interfaces the matrix array transducer elements 16 to the associated electronics such as ASICs 20 inside the probe handle, as discussed in greater detail below.

With this in mind, the illustrated ultrasound system 10 includes a transducer array 14 having transducer elements 16 suitable for contact with a subject or patient 18 during an imaging procedure. The transducer array 14 may be configured as a two-way transducer capable of transmitting ultrasound waves into and receiving such energy from the subject or patient 18. In such an implementation, in the transmission mode the transducer array elements 16 convert electrical energy into ultrasound waves and transmit it into the patient 18. In reception mode, the transducer array elements 16 convert the ultrasound energy received from the patient 18 (backscattered waves) into electrical signals.

Each transducer element 16 is associated with respective transducer circuitry, which may be provided as one or more application specific integrated circuits (ASICs) 20, which may be present in a probe or probe handle. That is, each transducer element 16 in the array 14 is electrically connected, as discussed herein, to a respective pulser 22, transmit/receive switch 24, preamplifier 26, swept gain 34, and/or analog to digital (A/D) converter 28 provided as part of or on an ASIC 20. In other implementations, this arrangement may be simplified or otherwise changed. For example, components shown in the circuitry 20 may be provided upstream or downstream of the depicted arrangement, however, the basic functionality depicted will typically still be provided for each transducer element 16. In the depicted example, the referenced circuit functions are conceptualized as being implemented on a single ASIC 20 (denoted by dashed line), however it may be appreciated that some or all of these functions may be provided on the same or different integrated circuits.

As also shown in FIG. 1, the transducer array 14 and ASIC(s) 20 are electrically interfaced via an interconnect structure 12, which may also be referred to herein as an electrical standoff. In accordance with present embodiments, the electrical standoff serves as an electrical interconnect and acoustic backing between the one or more integrated circuits (ASICs) 20 and the two-dimensional array 14 of ultrasound transducer elements, such as for use in real-time three-dimensional ultrasound imaging probes.

Also depicted in FIG. 1, a variety of other imaging components 30 are provided to enable image formation with the ultrasound system 10. Specifically, the depicted example of an ultrasound system 10 also includes a beam former 32, a control panel 36, a receiver 38, and a scan converter 40 that cooperate with the transducer circuitry to produce an image or series of images 42 that may be stored and/or displayed to an operator. A processing component (e.g., a microprocessor) and a memory of the system 10, such as may be present control panel 36, may be used to execute stored routines for processing the acquired ultrasound signals to generate meaningful images and/or motion frames, which may be displayed on a monitor of the ultrasound system 10.

With the preceding system level discussion in mind as useful context, the present approach, as noted above, provides for the manufacture and use of an electrical standoff which serves as an electrical interconnect and improved acoustic backing between one or more integrated circuits (ASICs) and a two-dimensional array of ultrasound transducer elements for real-time three-dimensional ultrasound imaging probes.

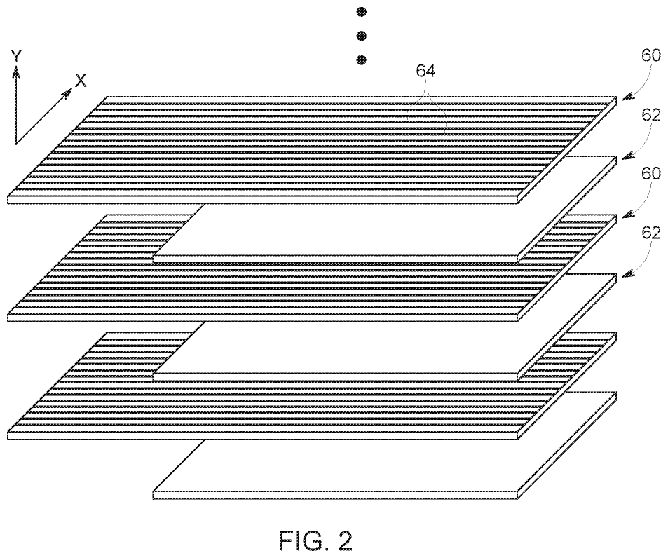

As noted above, in prior approaches, alternating sheets of flex circuits 60 and acoustic backing 62 (shown in a pre-lamination view in FIG. 2) are laminated together to form a “flex-plus-backing” stack that is post-processed to form a two-dimensional grid of interconnects in an x-y plane, in accordance with the depicted dimensions. One end of the laminated stack electrically connects to the matrix array of transducer elements, while the opposite end of the stack interfaces with the electrical elements within the probe handle. As described above, in this “flex-plus-backing” approach, the need for alternating layers of flex circuit (layers 60) and acoustic backing (layers 62) is costly and complicated and the protrusion of the relatively thick (e.g., ˜18 microns thick) low attenuation metal traces 64 on the flex circuits into the acoustic backing 62 degrades the acoustic performance.

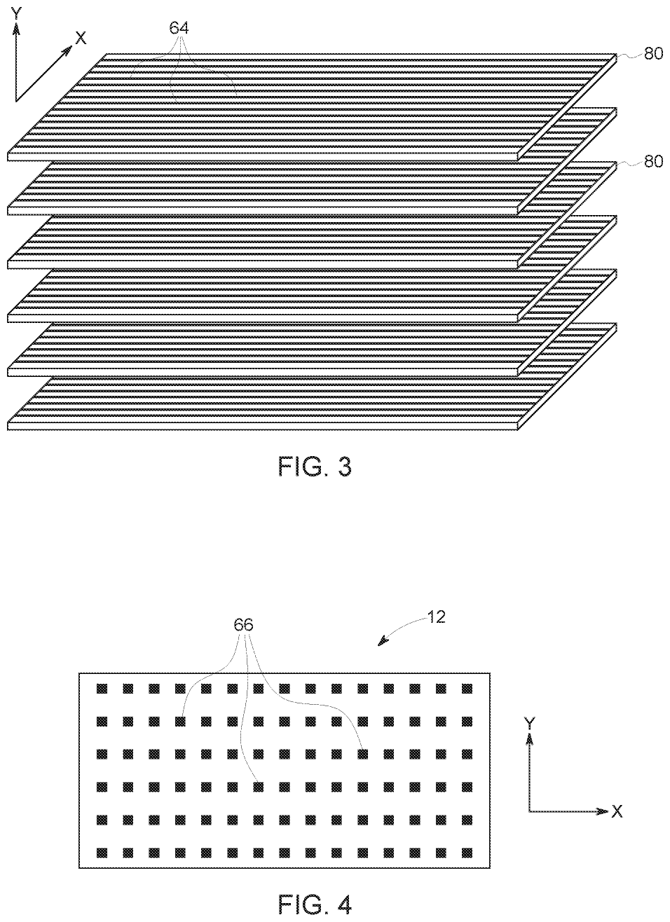

In contrast, and turning to FIG. 3, in accordance with the present approach the interconnect structure 12 (i.e., electrical standoff) that connects the transducer array 14 to the ASIC(s) 20 (either directly or indirectly via a separate flex interconnect circuit intermediary) is composed of sheets of acoustic backing 80 with printed traces 64 directly written or printed on the acoustic backing sheets 80, without separate flex circuit layers being incorporated as part of the standoff structure. To simplify explanation the present examples depict implementations in which each stacked acoustic backing sheet 80 with printed traces 64 is identical, though it may be appreciated that in different embodiments acoustic backing sheets within a given stack may vary from one another, such as in terms of the configuration of the printed traces 64. Examples, of suitable backing material for forming the acoustic backing sheets 80 include, but are not limited to, sheets of suitable epoxy materials in which scattering particles of different acoustic impedance (such as tungsten, microballoons, silicone beads) are embedded or otherwise disposed. The composition and structure of the acoustic backing providing substantial attenuation of acoustic energy. By way of example, the acoustic attenuation provided by the acoustic backing material may be greater than 2 db/(cm-MHz), such as approximately 10 dB/(cm-MHz), 20 dB/(cm-MHz) or, in certain implementations, between approximately 30 dB/(cm-MHz) to 50 dB/(cm-MHz).

The traces 64 may be printed on the acoustic backing sheet 80 using any suitable technique including, but not limited to: screen printing, ink-jet printing, aerosol jet printing, and so forth. By way of example, the acoustic backing material may be a sheet that is less than a millimeter thick, such as approximately 0.25 mm or 0.3 mm thick. The width of a trace 64 printed on the acoustic backing sheet 80, such as using aerosol jetting, may be less than a quarter of a millimeter, such as 0.1 mm, with a thickness of 10 microns or less, e.g., 5 microns, 3 microns, 2 microns, 1 micron, or 0.5 microns. In such an example, spacing between the printed traced 64 may be less than a quarter of a millimeter, such as approximately 0.12 mm. By using additive processes (e.g., printing) to form the traces 64, any desired thickness of the traces 64 may be obtained by tuning the printing parameters, in contrast to subtractive manufacturing processes. In this manner, the electrical and acoustic properties of the interconnect structure 12 may be adjusted.

The acoustic backing sheets 80 with printed traces 64 are laminated or adhered together to form an interconnect structure 12 having a two-dimensional array of contacts 66 (corresponding to the printed traces 64) on different surfaces, e.g., on opposing ends or differing faces, as shown on one end in FIG. 4 in an end-view. Thus, instead of an alternating layer structure, only acoustic backing layers 80 are present which are adhered or otherwise affixed to one another. As may be appreciated, pitch of the conductive material forming the trace 64 in the x-direction is determined by the spacing of the traces 64 as printed on the individual acoustic backing sheets 80. Pitch of the conductive material forming the trace 64 in the y-direction is determined by the thicknesses of the acoustic backing sheets 80.

With respect to acoustic properties of the interconnect structure 12, the thickness of the traces 64 printed directly on the acoustic backing sheets 80 may be less (e.g., 0.5 microns to 10 microns) than the thickness typically present in a flex circuit layer (typically ˜18 microns), resulting in the volume fraction of acoustic backing in the total interconnect structure being higher than what is obtained in an interleaved flex circuit layer-acoustic backing layer structure. As a result, the interconnect structure 12 may have superior acoustic properties in comparison to a structure having interleaved flex circuit layers. To further improve acoustic performance, conductive polymer inks (such as polyanilines, polypyrrols, or polythiophenes inks or inks based on silver coated polymer particles) may be used to print conductive traces, which may enhance the acoustic properties and improve acoustic attenuation.

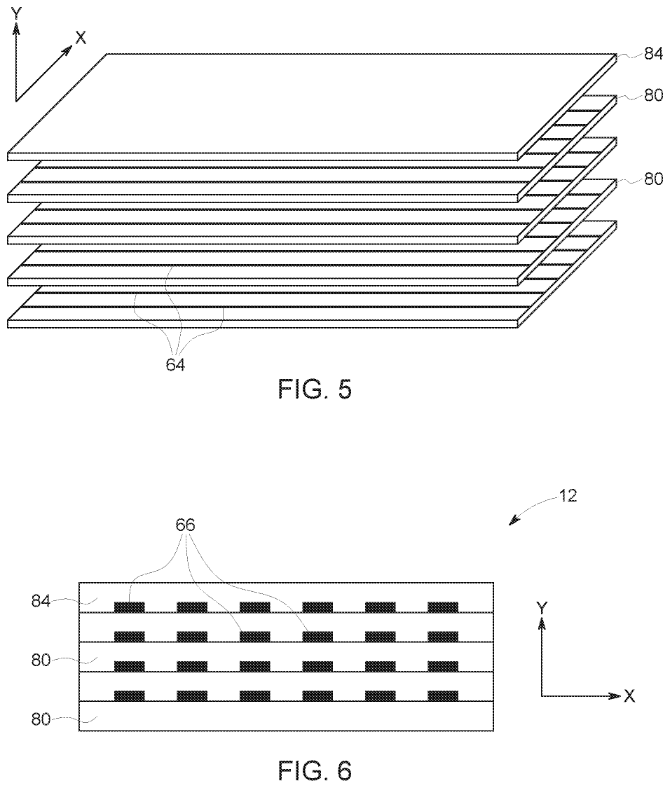

As may be appreciated, in certain embodiments certain of the sheets or layers of acoustic backing material may not have traces 64 printed on them. For example, a terminal layer (i.e., topmost and/or bottommost layer) may be blank, (i.e., have no printed traces 64) to avoid having exposed traces on a surface of the laminated interconnect structure 12, An example of this is shown in FIGS. 5 (exploded perspective view) and 6 (end view), where a topmost acoustic backing sheet 84 is blank (i.e., has no printed traces 64) so that the top surface of the interconnect structure 12 does not include exposed traces 64. Further, it may be appreciated that, instead of or in addition to a blank acoustic backing sheet 84 being added as the topmost and/or bottommost layer, in certain implementations a metal sheet or layer may be added to the top or bottom of the stack, such as to provide electromagnetic shielding.

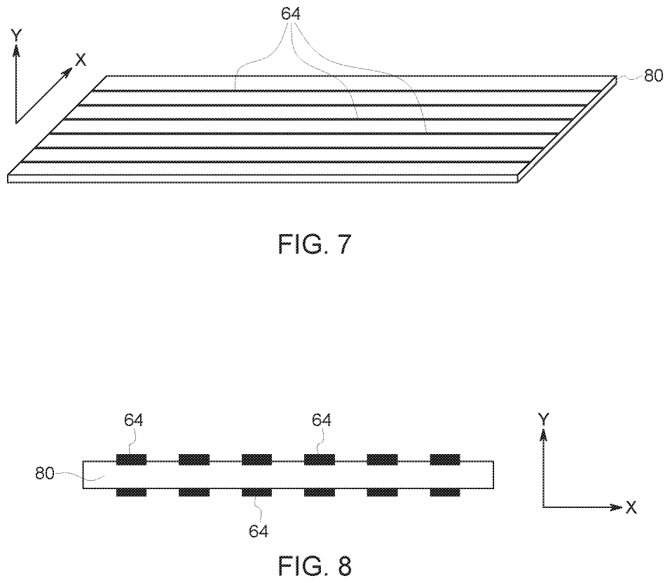

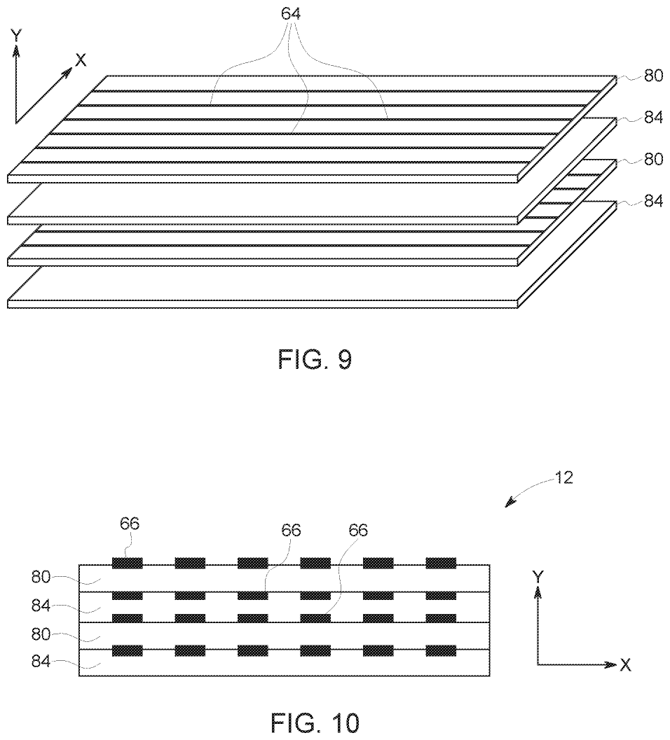

With this in mind, FIGS. 7-10 provide examples of a further embodiment in which certain of the acoustic backing layers 80 have traces 64 printed on both sides (i.e., opposing surfaces. This is shown in a perspective and end view for a single acoustic backing sheet 80 in FIGS. 7 and 8. As shown in FIGS. 9 (exploded perspective view) and 10 (end view), a laminated interconnect structure 12 may be fabricated by alternating acoustic backing sheets 80 having traces 64 printed on opposing surfaces with blank or unprinted acoustic backing sheets 84 to form a laminated interconnect structure 12.

While conductive inks may be used in printing some or all of the traces 64, in addition, in certain embodiments the ink or inks may be used to provide different or varying electrical properties. For example, different inks can be printed on the acoustic backing sheet(s) 80 to create a variety of different impedances for the electronic signals transmitted via the resulting printed traces. For example, conductive and resistive (carbon, graphite and carbon/graphite blends, etc.) inks can be printed as parallel traces on one or more acoustic backing sheets 80, which may allow for the paired traces to be used for operating compound or complex transducer arrangements in a controlled or tuned manner. Similarly, resistive ink or properties may be added to certain of the printed traces 64 to facilitate apodization, and thereby reduce side lobes in ultrasound beams.

Further, more complex electrical structures may be printed on some or all of the acoustic backing sheets 80. For example, keeping in mind that the ink(s) used in forming such structures may be provide a variety of electrical properties, passive circuit elements (e.g., inductive, resistive, and/or capacitive elements) may be incorporated into the features (e.g., traces 64) printed onto the acoustic backing layers 80. For example, to improve efficiency transducer arrays 14 may benefit from electrical tuning based on parallel or series inductance. In accordance with the present approach, this may be accomplished by adding inductance in the printed traces 64 running to the transducer elements 16.

In addition to providing improved acoustic performance (i.e., effective attenuation of unwanted acoustic energy traveling towards the backing), the interconnect structure 12 may also improve thermal performance-isolation of heat dissipating electronics from the patient contact surface resulting in a lower temperature of the patient contact surface. That is, the interconnect structure 12 (e.g., an electrical standoff) may provide acoustic isolation in conjunction with improved or increased thermal resistance between the power-dissipating ASIC 20 and the patient 18, reducing the temperature at the patient contact surface. For example, the acoustic backing material may also function as or be recreated with thermal enhancements to create a thermally insulating material, which may act to increase the thermal resistance between the patient contacting surface of the probe and the heat-generating ASIC(s) which may also be present in the probe. In this manner, the temperature of the patient contacting surface may be reduced or otherwise limited. By way of example, the acoustic backing material may have a thermal conductivity of less than or equal to 1 W/(mK).

Further, the interface structure may allow for a reduced probe footprint at the patient contact surface, which results in better access to acoustic windows for imaging the body. Other desirable properties of an interconnect structure formed in this manner include, but are not limited to: a probe footprint at the patient contact surface that is independent of the ASIC and ASIC interconnect size, the potential for pitch independence between the ASICs connections and the transducer matrix array, and suitability for lower cost mass production.

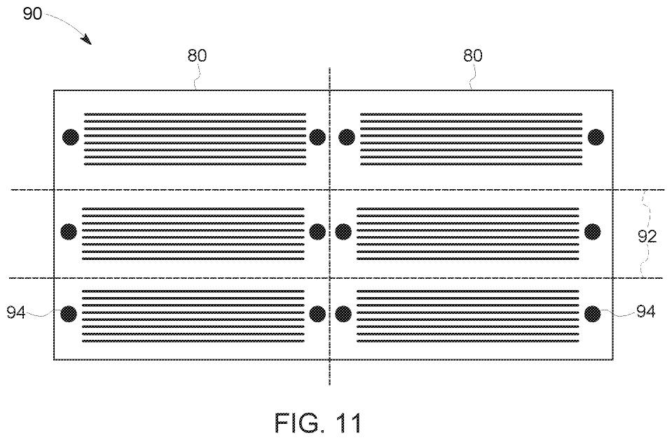

Turning to FIG. 11 in certain implementations it may be desirable to print multiple acoustic sheets 80 on a larger sheet 90 that may then be cut (such as along dashed lines 92) so as to form the acoustic backing sheets 80 to be used in the interconnect structure 12. Such an approach may provide for greater manufacturing efficiencies of scale, such as allowing for printing on a sheet of the acoustic material at a commercially available size and/or allowing for a more optimal use of a printing device that is capable of printing on the larger sheet 92 in one operation as opposed to performing multiple, separate printing operations to print to the smaller size sheets.

It may be noted that the example of FIG. 11 depicts each smaller acoustic backing sheet 80 to be cut from the larger sheet 90 as including alignment features 94, here depicted as printed fiducials corresponding to where paired tooling holes will be formed. A mechanical or laser drilling process may then utilize the fiducials to subsequently form an alignment hole and/or slot used to align a stack of acoustic backing sheets 80. Alternatively, the alignment features may be pre-formed in the larger sheet 90 prior to printing the traces 64. Alignment features 94 so formed may be used to facilitate precise alignment of acoustic backing sheets 80 in an interconnector structure assembly step.

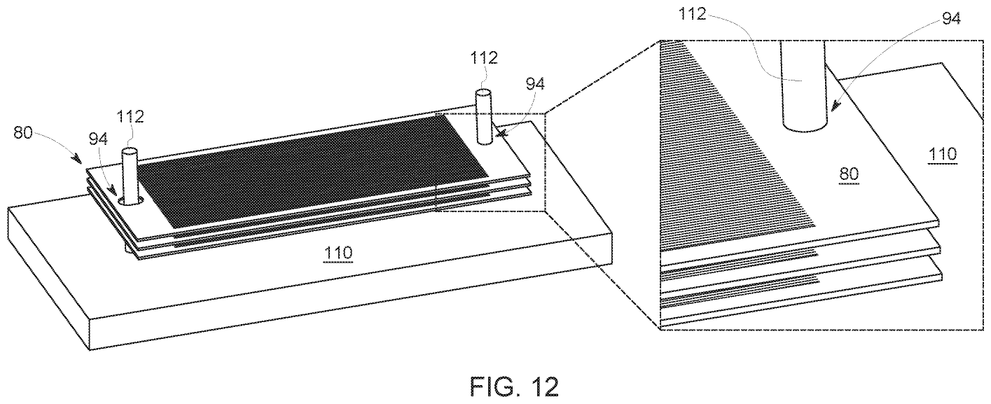

For example, turning to FIG. 12 a tooling fixture 110 is shown on which acoustic backing sheets 80 having alignment features 94, in the form of tooling holes and slots, are positioned. In particular, in this example the tooling fixture 110 includes tooling pins 112 which pass through the tooling holes and slots to allow a precisely aligned stack of acoustic backing sheets 80 to be formed. The aligned stack of acoustic backing sheets 80 may be laminated to form the interconnect structure 12.

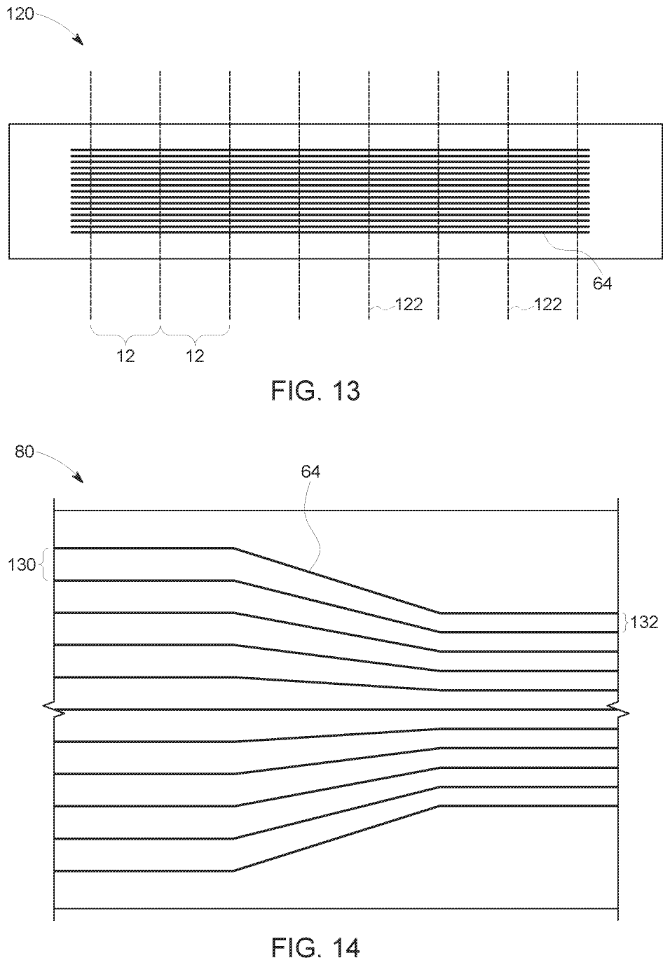

In a further manufacturing implementation shown in FIG. 13, it may be appreciated that a laminated structure 120 that is larger than the desired interconnect structures 12 may be initially formed, such as by laminating a stack of printed acoustic backing layers together where the laminated sheets are larger than the lengths of the acoustic backing layers from which the interconnect structure 12 is made. The laminated structure 120 may then be cut at precise lengths, as shown in FIG. 13 at dashed lines 122, to form the laminated structures corresponding to the interconnect structures 12. In this manner, as with the preceding example, greater efficiency and/or cost reduction may be achieved by fabricating an initial structure better suited or sized with respect to the manufacturing equipment or materials, with a subsequent step or step being employed to size an initially formed structure to what is suitable for use in or as the interconnect structure 12.

for simplicity of illustration, the preceding two implementations are presented in the context of a flat or straight cut to either the acoustic sheets 80 or the laminated structure 120. However, it should be appreciated that in practice, one or more surfaces or edges of such structures may be cut at an angle or curve (e.g., convex or concave) based on the geometry of the enclosure and/or the geometry of the electrical structure to which a connection is being formed. For example the transducer array 14 may in practice have a convex shape to better conform to patient anatomy. In such an example, the corresponding facing surfaces of the electrical standoff formed in accordance with the present approach may be correspondingly curved.

Turning to FIG. 14, in further implementations, and in reference to an aspect noted above, the pitch or spacing of the conductive traces at different ends of the acoustic backing sheets 80 may differ, such that the spacing 130 between printed traces at one end is different than the spacing 132 at the other end. In this manner, the spacing of the printed traces 64 at one end of the acoustic backing sheet 80 relative to the other end may vary to suit a particular application. As will be appreciated, a laminated stack of such acoustic backing sheets 80 yields an interconnect structure 12 with different pitch or spacing of the conductive interconnects at each end, so that the spacing at one end may better correspond to the spacing of the connections at an ASIC 20 and the connections at the other end correspond to the spacing of the connections at the transducer array 14. Thus, a single ASIC 20 could be fabricated with element connections at a particular spacing, and an electrical standoff fabricated using acoustic backing sheets 80 as shown in FIG. 14 could be used to adapt the same ASIC 20 for many different probe applications that require different element spacings.

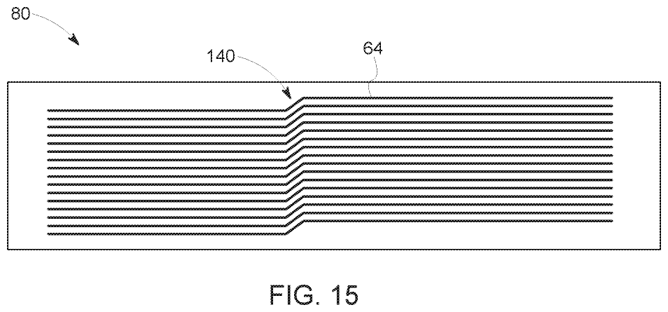

Turning to FIG. 15, a further aspect of using non-linear conductive printed traces 64, here depicted as traces 64 in which one or more angled turns 140 is present, is the possible improvement in acoustic performance. In particular, in one embodiment the traces 64 may be printed so as to introduce at least one angled turn 140 so that the path of the traces 64 is not entirely linear. Correspondingly, the path through the acoustic backing material between traces 64 is lengthened in that the acoustic waves propagating between the traces 64 are reflected at the angled turns 140 in the traces 64, thereby attenuating these acoustic waves to a greater extent by causing the waves to travel a greater distance through the acoustic backing material, and thereby improving acoustic performance.

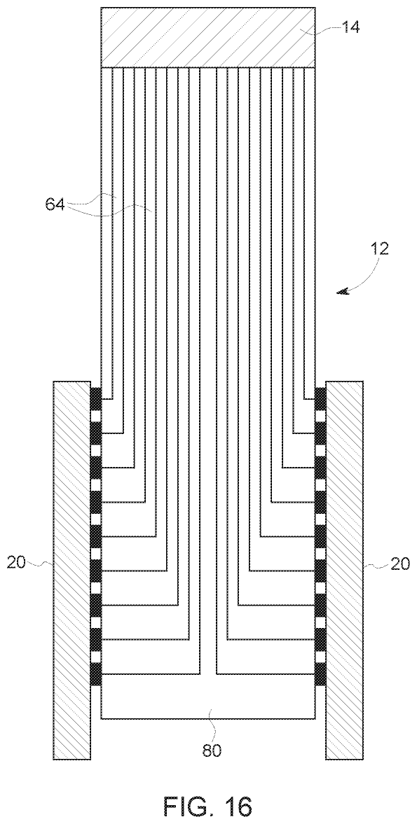

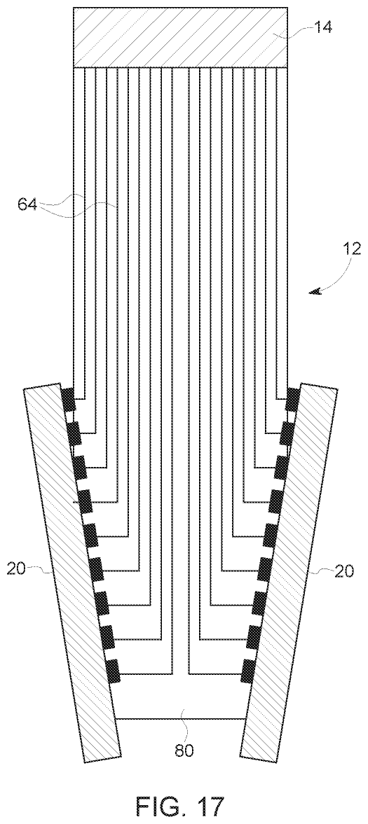

While the preceding examples illustrate electrical standoffs in which the contacts 66 are formed on opposing surfaces, it should be appreciated that in certain embodiments the contacts, and corresponding coupling to electrical components or circuits, may be on non-opposing surfaces of the electrical standoff formed as a laminated interconnect structure 12. For example, FIGS. 16 and 17 depict examples of an acoustic backing sheet 80 of an electrical interconnect 12 in which the connection to electrical components (here a transducer array 14 and ASICs 20) are made at non-opposing faces of the interconnect structure 12.

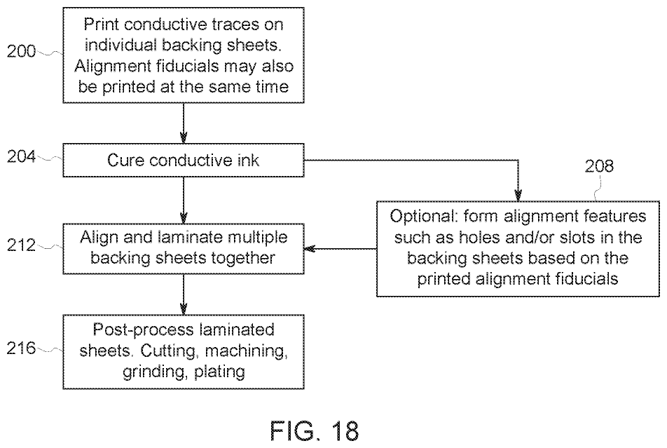

With the preceding in mind, and turning to FIG. 18, a flowchart is depicted describing steps in the manufacture of an interconnect structure 12 consistent with the preceding discussion. With reference to this process flow, a flowchart is depicted describing steps in the manufacture of an interconnect structure 12 consistent with the preceding discussion. With reference to this process flow, at step 200 printed traces 64 are printed on individual acoustic backing sheets 80, as discussed herein. If alignment fiducials are to be employed, the fiducials may also be printed at step 200. As noted herein, the printing technique may be screen printing, ink jet printing, aerosol jet printing, or another suitable printing technique.

Once printed, the printed traces 64 may be cured (step 204) using an approach suitable to the printing technique and ink type. Examples of suitable curing techniques include, but are not limited to: heating (such as in an oven), laser sintering, photonic sintering, and so forth.

In an optional step 208, alignment features (e.g., holes, slots, and so forth) may be formed in the acoustic backing sheets 80, such as based on printed alignment fiducials or other precision machining techniques.

Using the alignment features (or other alignment techniques), the acoustic backing sheets 80 on which printed traces 64 are printed are aligned and stacked (step 212) so as to form aligned stacks of sufficient sheets 80 to form the interconnect structure 12 being manufactured. The aligned stacks of acoustic backing sheets 80 are laminated to form the interconnect structure.

A post-processing step 216 may be performed on the laminated stack of sheets as a final step in forming the interconnect structure 12. Examples of post-processing operations include, but are not limited to cutting, machining, grinding, and so forth. Contacts on the component facing surfaces of the interconnect structure 12 may be formed as part of the post-processing step or after this step by plating, screen printing, ink-jet printing, or aerosol jet printing the respective contact structures at the proper locations on the component facing surfaces.

As previously noted, other steps not shown in FIG. 18 may also occur. For example, to the extent that multiple acoustic backing sheets are printed on a single larger sheet, a separate cutting step may be performed prior to the act of aligning and laminating the backing sheets together. Similarly, to the extent that a larger laminated structure is formed which will be cut into multiple interconnect structures 12, part of post-processing step 216 may be to cut the laminated structure into the target interconnect structures 12.

In addition, though the preceding discussion and examples relate to the forming an interconnect structure by laminating acoustic backing sheets 80 on which printed traces 64 are printed, it should be appreciated that the interconnect structure 12 (such as shown in FIG. 4) may be integrally formed using an additive manufacturing process (such as a 3D printing process), without the intermediary steps of processing and laminating separate acoustic backing sheets 80. In such an additive manufacturing process, the entirety of the interconnect structure 12 may be printed so as to form an integral structure, without going through an intermediary step of printing on acoustic backing sheets.

Technical effects of the invention include an electrical interconnect structure composed of sheets of acoustic backing with conductive traces directly written or printed on the acoustic backing sheets, without a separate flex circuit layer. The acoustic backing sheets with conductive traces are laminated together to form the interconnect structure, which has a two-dimensional array of interconnects corresponding to the conductive traces. Thus, instead of an alternating layer structure of flex circuit layer interleaved with acoustic backing layers, only acoustic backing layers are present.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An electrical standoff, comprising:
a plurality of acoustic backing layers, wherein the acoustic backing layers are stacked on one another to form a laminated structure; and
a plurality of conductive traces formed on one or more of the acoustic backing layers, wherein the conductive traces form conductive contacts on different surfaces of the laminated structure for coupling two electrical components.

2. The electrical standoff of claim 1, wherein the two electrical components comprise an ultrasound transducer array and driving and receiving circuitry.

3. The electrical standoff of claim 1, wherein the acoustic backing layers are adhered to one another without an intervening layer separating them.

4. The electrical standoff of claim 1, wherein the plurality of conductive traces comprises conductive ink printed on each acoustic backing layer.

5. The electrical standoff of claim 1, further comprising one or more resistive traces printed on some or all of the acoustic backing layers.

6. The electrical standoff of claim 1, further comprising one or more passive circuit elements formed on some or all of the acoustic backing layers and in communication with the conductive traces.

7. The electrical standoff of claim 1, wherein at least some of the conductive traces formed on each acoustic backing layer do not form a straight line from a first end of the acoustic backing layer to a second end.

8. The electrical standoff of claim 1, wherein at least some of the conductive traces formed on each acoustic backing layer include an angled turn such that spacing between the conductive traces at a first end of a respective acoustic backing layer differs from spacing between the conductive traces at a second end of the respective acoustic backing layer.

9. The electrical standoff of claim 1, wherein at least some of the conductive traces formed on each acoustic backing layer include at least one angled turn such that the path between pairs of conductive traces on a respective acoustic backing layer does not provide a linear path from a first end of the respective acoustic backing layer to a second end of the respective acoustic backing layer.

10. The electrical standoff of claim 1, wherein a thickness of the conductive traces formed on each acoustic backing layer is less than 10 microns.

11. The electrical standoff of claim 1, wherein one or more of the acoustic backing layers has a thermal conductivity of less than 1 W/(mK).

12. The electrical standoff of claim 1, further comprising one or more blank acoustic backing layers on which conductive traces are absent.

13. The electrical standoff of claim 1, wherein one or more of the acoustic backing layers comprise conductive traces formed on opposing surfaces of the respective backing layers.

14. A method for forming an electrical standoff structure, comprising the acts of:
printing conductive ink to form conductive traces on one or more acoustic backing layers of a plurality of acoustic backing layers;
stacking the plurality of acoustic backing layers; and
laminating the plurality of acoustic backing layers to form a laminated structure used to form the electrical standoff structure.

15. The method of claim 14, further comprising:
printing one or more alignment fiducials on each acoustic backing layer;
forming one or more alignment features in each acoustic backing layers using the printed alignment fiducials; and
using the one or more alignment features to stack the plurality of acoustic backing layers.

16. The method of claim 14, further comprising:
curing the conductive ink after printing.

17. The method of claim 14, wherein no additional layers are interleaved with the acoustic backing layers when stacking the plurality of acoustic backing layers.

18. The method of claim 14, further comprising:
processing the laminated structure to form the electrical interconnect structure.

19. The method of claim 18, wherein processing the laminated structure comprises one or more of cutting, machining, or grinding, the laminated structure and forming contacts by one or more of plating, screen printing, ink-jet printing, or aerosol jet printing to form the electrical interconnect structure.

20. The method of claim 14, wherein printing conductive ink to form conductive traces on each acoustic backing layer comprises printing the conductive ink corresponding to each acoustic backing layer on one or more backing sheets that are each larger than the respective acoustic backing layers; and further comprising:
cutting the one or more backing sheets to form the acoustic backing layers.

21. An ultrasound probe, comprising:
a transducer array comprising a plurality of separate transducer elements;
driving and receiving circuitry configured to communicate with each individual transducer element; and
an electrical standoff structure electrically connecting the driving and receiving circuitry for each transducer element to each respective transducer element; wherein the electrical standoff structure comprises a laminated stack of acoustic backing layers on which conductive traces are formed on at least a portion of the acoustic backing layers.

22. The ultrasound probe of claim 21, wherein the driving and receiving circuitry for each transducer element is provided as an application specific integrated circuit (ASIC).

23. The ultrasound probe of claim 21, wherein the laminated stack of acoustic backing layers are not separated by other layers.

24. The ultrasound probe of claim 21, wherein at least some of the conductive traces formed on each acoustic backing layer include an angled turn such that spacing between the conductive traces at a first end of electrical interconnect structure differs from spacing between the conductive traces at a second end of the electrical interconnect structure.

25. The ultrasound probe of claim 21, wherein at least some of the conductive traces formed on each acoustic backing layer include at least one angled turn such that the path between pairs of conductive traces on a respective acoustic backing layer does not provide a linear path from a first end of the electrical interconnect structure to a second end of the electrical interconnect structure.
